(19) United States
(12) Patent Application Publication
(10) Pub. No.: US 2023/0210731 A1
(43) Pub. Date: Jul. 6, 2023

(54) REDUCTION RESISTANT HYDROGEL THICKENERS

(71) Applicant: CAO Group, Inc., West Jordan, UT (US)

(72) Inventor: Steven D. Jensen, South Jordan, UT (US)

(73) Assignee: CAO Group, Inc., West Jordan, UT (US)

(21) Appl. No.: 17/568,707

(22) Filed: Jan. 4, 2022

(51) Int. Cl. A61K 8/04; A61K 8/23; A61K 8/88; A61K 8/81; A61Q 11/00

(52) U.S. Cl. CPC A61K 8/042; A61K 8/23; A61K 8/88; A61K 8/8188; A61Q 11/00

(57) ABSTRACT

A reductive composition, suitable for the dental industry, which utilizes sodium dithionite may be provided when it is combined with either polyethyloxazoline or polyvinylpyrrolidone. Other dithionite molecules, such as potassium dithionite, may be used. Likewise, the methods described herein may be applied to other reductive agents.

### FIELD OF THE INVENTION

The present invention relates to the field of dentistry and more particularly relates to the use of reduction resistant thickeners in tooth whitening compounds to create novel products.

### BACKGROUND OF THE INVENTION

Traditional tooth whitening compounds utilize oxidation reactions to whiten teeth, usually in the form of introducing peroxides to the enamel surface. These peroxide-based teeth whitening systems whiten by oxidizing the extrinsic and intrinsic stains found in teeth by removing electrons from the chromophores of stains, rendering them white. It should be noted that oxygen is not necessary for this process, however oxygen is notorious for initiating it, thus the element provides the name for the process. Hydrogen peroxide and its derivatives are stable in acidic pH environments and become increasingly unstable as the pH nears neutrality (pH 7). Bulk hydrogen peroxide is usually stored at a pH below 2. Acidic teeth whitening systems are detrimental to tooth enamel as they etch and dissolve tooth structure. Therefore, it is imperative that peroxide based whitening systems have at least a neutral pH. This is one of the main shelf-life instability factors with respect to peroxide-based tooth whiteners and the reason these compositions degrade over a short period of time. However, these whitening compounds also oxidize tooth enamel, resulting in tooth etching. As such, oxidative whitening of teeth is significantly regulated outside the United States and Canada.

Reduction reactions could also provide whitening effects. Reduction is the opposite reaction of oxidation in that electrons are added to a molecule and is often seen in conjunction with oxidation reactions (together being called redox reactions). Reduction of the chromophores of intrinsic and extrinsic enamel stains also renders them white. However, powerful reducing agents present an interesting challenge with respect to reactivity and compatibility when formulating various compositions. What usually occurs is an immediate reaction that results in a permanently reduced ingredient. For example, sodium dithionite (also known as sodium hydrosulfite, shown in the FIG. 1) is a powerful reducing agent commonly used in dying processes, water treatment, gas purification, photography, and other industries. It has not been previously used in dental whitening products. Sodium dithionite is highly reactive in the presence of water, quickly decomposing to thiosulfates and bisulfates when so exposed.

The present invention is a stable composition formulated from a combination of a reduction agent and appropriate hydrogels to form said compositions. The present invention represents a departure from the prior art in that the compositions formulated using the teachings of the present invention allow for the use of highly reductive agents in wider industries, such as dentistry.

### SUMMARY OF THE INVENTION

Polyethyloxazoline and polyvinylpyrrolidone (PVP) are surprisingly resistant to the reduction potential of dithionite. Aqueous compositions made with these thickeners resist dithionite decomposition even in formulas that contain substantial water, which accelerates the reaction of dithionite. Dried hydrogels made with polyethyloxazoline and PVP are especially good at preserving dithionite stability when the water content is below 25% and produce a shelf-stable product that is deliverable to a customer with sufficient active ingredient to be effective. The added stability that hydrogels provide dithionite solutions is that they physically displace water in the final composition or product. As such, polyethyloxazoline or PVP may be used as a gelling agent that resists the natural reduction of sodium dithionite in the presence of water while producing a consumer whitening product.

In view of the foregoing disadvantages inherent in the known types of dental whitening products, an improved dental whitening composition may provide a chemically reductive dental composition utilizing sodium dithionite that meets the following objectives: that it has a reasonable shelf life, safe, and effective for a consumer’s use, inexpensive to formulate and package, and that use of the final product would be familiar to end consumers. As such, a new and improved reductive composition may comprise a blend of sodium dithionite with either PVP or polyethyloxazoline to accomplish these objectives.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

To describe the way the above-recited and other advantages and features of the invention can be obtained, a more particular description of the invention briefly described above will be rendered by reference to specific example embodiments thereof which are illustrated in the appended drawings. Understanding that these drawings depict only typical embodiments of the invention and are therefore not to be considered as limiting of its scope, the invention will be described and explained with additional specificity and detail using the accompanying drawings.

FIG. 1 is a drawing of a sodium dithionite molecule (prior art).

FIG. 2 is a schematic drawing showing base gel formation.

FIG. 3 is a schematic drawing showing the addition of the reductive agent top the gel.

FIG. 4 is a schematic drawing showing the final reductive compound.

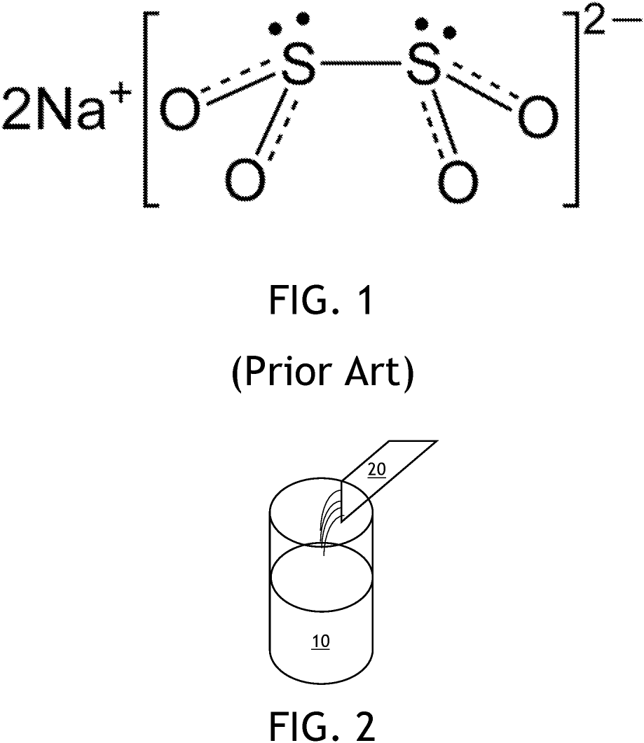

### DESCRIPTION OF THE PREFERRED EMBODIMENT

Preferred embodiments of the chemically reductive compositions are herein described. It should be noted that the articles “a”, “an”, and “the”, as used in this specification, include plural referents unless the content clearly dictates otherwise.

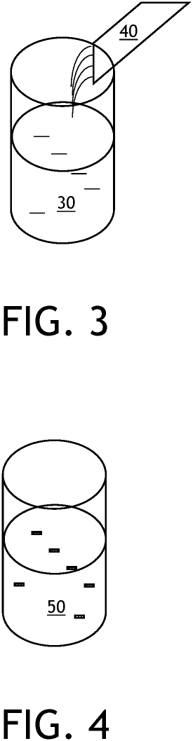

In preparation of a gel from reductive agents, particularly the salts of dithionite, it is important to first form a carrier gel (FIG. 2) by combining the thickener 20 into water 10. The salts of dithionite are notoriously unstable in water, so by creating the gel first, the dithionite retains more of its active properties and the whole process is more stable. Once the gel 30 thickens (FIG. 3) the salts of dithionite 40 are added, yielding an active reduction compound 50 (FIG. 4). The salts of dithionite are usually added in powder form and mixed with the carrier gel. It is important to note that either the sodium or potassium salt of dithionite may be used, but the sodium salt is preferred. The preferred thickeners may be chosen from either PVP or polyethyloxazoline, though any later discovered polymeric thickener with equal or superior stability while in contact with dithionite would be considered an equivalent. The gel stability is unexpected as no other thickeners have demonstrated any significant resistance to the reductive capacity of a dithionite in water. However, for best results, it is found that keeping the water content at or below 25% of the gel formulation by weight improves dithionite stability.

Although the present invention has been described with reference to preferred embodiments, numerous modifications and variations can be made and still the result will come within the scope of the invention. The described embodiments are to be considered in all respects only as illustrative and not restrictive. No limitation with respect to the specific embodiments disclosed herein is intended or should be inferred. Therefore, the scope of the invention is indicated by the appended claims rather than by the foregoing description. All changes which come within the meaning and range of equivalency of the claims are to be embraced within their scope.

What is claimed is:

1. A chemically reductive composition comprising a dithionite molecule interspersed in a gel that further comprises a gelling agent selected from the set of gelling agents consisting of polyethyloxazoline and polyvinylpyrrolidone.

2. The chemically reductive composition of claim 1, the dithionite molecule being chosen from the set of dithionite molecules consisting of sodium dithionite and potassium dithionite.

3. A chemically reductive composition comprising a chemically reductive agent interspersed in a gel that further comprises a gelling agent selected from the set of gelling agents consisting of polyethyloxazoline and polyvinylpyrrolidone.
